(19) United States
(12) Patent Application Publication
(10) Pub. No.: US 2005/0175552 A1
(43) Pub. Date: Aug. 11, 2005

(54) Tooth coating compositions and methods therefor

(76) Inventors: Diego Andres Hoic, Jersey City, NJ (US); John P. Curtis, Alpha, NJ (US)

Correspondence Address: HARNESS, DICKEY, & PIERCE, P.L.C, 7700 BONHOMME, STE 400, ST. LOUIS, MO, 63105, US

(21) Appl. No.: 10/775,706

(22) Filed: Feb. 10, 2004

(51) Int. Cl. A61K 7/16

(52) U.S. Cl. 424/49

(57) ABSTRACT

A tooth coating comprising at least one resin, at least one colorant, and pearlescent particles are disclosed. The resin can be a natural resin or a synthetic polymer. Methods of coloring a tooth are also disclosed.

### FIELD

This invention relates to compositions and methods for whitening or coloring teeth. The invention also relates to compositions and methods for applying active components such as pharmaceutically active compounds, anti-caries agents, or breath fresheners to teeth.

### BACKGROUND

Consumers wishing to whiten or color their teeth have a limited variety of products from which to choose. Successful application of some products, such as veneers, crowns and caps, involves destruction of tooth enamel, and requires the services of a dental professional such as a dentist. Furthermore, tooth whitening or coloring products have not been used as carriers for active materials such as pharmaceutically active compounds, anti-caries agents, or breath fresheners. Thus, there is an ongoing need for new compositions and methods for whitening teeth, in particular compositions and methods for whitening teeth without destruction of tooth enamel, as well as compositions and methods for delivering active materials to the oral cavity.

### SUMMARY

Accordingly, the present inventors have succeeded in developing compositions and methods for imparting whiteness or color to teeth. A composition of the present invention can be a tooth coating comprising at least one natural or synthetic resin and at least one colorant. Application of a composition to the teeth in a mammal such as, for example a human, coats the teeth and imparts a whiteness or a color to the teeth.

Thus, in various embodiments, the present invention can involve a tooth coating. In certain embodiments, the tooth coating can be a tooth-coating fluid, a veneer, a natural hard dental surface, an artificial hard dental surface, a crown, a restorative veneer, a partial denture, or a composite. In various embodiments, the tooth coating of the present invention can comprise at least one film-forming, synthetic polymer at a concentration of at least 10% (w/w), a colorant, and pearlescent particles.

The invention, in various embodiments, can also include a tooth coating comprising at least one film-forming resin, a colorant at a concentration of at least about 1% (w/w), and pearlescent particles at a concentration of at least about 3% (w/w).

Methods of coloring a tooth in a mammal are also encompassed in various embodiments of the present invention. The methods can comprise applying to the tooth a tooth-coloring amount of a tooth coating comprising at least one film-forming synthetic polymer at a concentration of at least 10% (w/w), a colorant, and pearlescent particles.

In various embodiments, the methods can also comprise applying to the tooth, a tooth-coloring amount of a fluid comprising at least one film-forming resin, a colorant at a concentration of at least about 1% (w/w), and pearlescent particles at a concentration of at least about 3% (w/w).

In various embodiments the film-forming resin can be one or more synthetic resins, natural resins or combinations thereof. The synthetic resins can be polyacrylates, polyacrylamides, polyvinylpyrollidones, polyvinylalcohols, polyethylene glycols, polyethylene oxides, polystyrenes, hydroxypropyl methyl celluloses, hydroxypropyl celluloses, hydroxyethyl celluloses, methyl celluloses, carboxymethyl celluloses, copolymers, block copolymers, graft copolymers cross-linked polymers or combinations thereof. The natural resin can be colophoniums, mastics, shellacs, natural celluloses or combinations thereof.

In various embodiments of the present invention, the colorant can comprise one or more types of white particles. Such white particles can independently comprise a material selected from the group consisting of titanium oxide, aluminum oxide, tin oxide, calcium oxide, magnesium oxide, zinc oxide, polyethylene, polypropylene, ethylene/propylene copolymer, polytetrafluoroethylene, and polyhexafluoropropene. In various embodiments, the colorant can also comprise one or more types of non-white particles. Such non-white particles can independently comprise a material selected from the group consisting of copper oxide, chromium oxide, ferric oxide, ultramarine blue, phthalocyanine green, FD&C Green No. 1 lake, FD&C Blue No. 2 lake, D&C Red No. 30 lake, FD&C Yellow No. 15 lake, FD&C Red No. 3, D&C Red No. 30, Food Red No. 17, disodium salt of 6-hydroxy-5-{(2-methoxy-5-methyl-4-sulphophenyl)azo}-2-naphthalenesulfonic acid, Food Yellow No. 13, sodium salt of a mixture of the mono and disulphonic acids of quinophtalone or 2-(2-quinolyl) indanedione, FD&C Yellow No. No. 5, FD&C Yellow No. 6, FD&C Green No. 3, FD&C Blue No. 1, FD&C Blue No. 2, and combinations thereof.

In various embodiments, the pearlescent particles can comprise one or more types of particles which are independently comprised of a material selected from the group consisting of bismuth oxychloride, nacre, mollusk shell, fish scale, pearl, silicates selected from the group consisting of mica, opal and silica, and combinations thereof. Such pearlescent particles can be coated with a metal oxide selected from the group consisting of titanium oxide, iron oxide, aluminum oxide, tin oxide, copper oxide, calcium oxide, magnesium oxide, barium oxide, chromium oxide and combinations thereof. In some configurations, the pearlescent particles can further comprise a protective coating. A protective coating can protect the particles from reacting chemically with other components of the composition, or vice versa. In non-limiting example, a metal oxide such as titanium oxide comprised by a pearlescent particle could be chemically reactive towards a colorant such as an organic dye colorant; contact between the metal oxide and the dye could lead to degradation of the dye. The presence of a coating on the particle can prevent such degradation. In some configurations, the protective coating can be a transparent coating or a translucent coating. A transparent coating or translucent coating can allow the particles to retain their pearlescent appearance.

In various embodiments of the present invention the colorant can be titanium dioxide at a concentration of at least about 1% (w/w), the pearlescent particles can be mica titanium at a concentration of at least about 3% (w/w) and the synthetic polymer can be a polyacrylate.

In various embodiments, the compositions of the present invention can further comprise an active ingredient selected from the group consisting of anti-caries agents, anti-sensitivity agents, anti-microbial agents, bleaching agents, and combinations thereof.

### DETAILED DESCRIPTION

The present invention includes compositions and methods for imparting whiteness or color to teeth. The compositions can be tooth coatings comprising at least one natural or synthetic resin and at least one colorant. The colorant can impart whiteness or a non-white color. The compositions can further comprise pearlescent particles. Application of the compositions to the teeth in a mammal such as, for example a human, coats the teeth and imparts a whiteness or a color to the teeth.

In certain configurations, the tooth coating can comprise at least one film-forming resin. The total film-forming resin concentration can be from at least about 10% (w/w) up to about 95% (w/w), from at least about 15% (w/w) up to about 90% (w/w), from at least about 20% (w/w) up to about 85% (w/w), from at least about 25% (w/w) or 26% (w/w) up to about 60% (w/w), 70% (w/w), or 80% (w/w), or from at least 30% (w/w) up to about 60% (w/w). A film-forming resin can be a natural resin or a synthetic resin, or a combination thereof. In certain configurations, a natural resin can be selected from the group consisting of colophoniums, mastics, shellacs and combinations thereof. In certain configurations, a tooth coating of the invention can comprise at least one natural resin and at least one synthetic polymer. In certain configurations, the tooth coating can comprise a colophonium, a mastic, and a polyacrylate.

In some embodiments, the film-forming resin can comprise a synthetic polymer. The term “polymer,” as used herein, can encompass both acid and salt forms of homopolymers, copolymers, block copolymers, graft copolymers, and cross-linked polymers. Non-limiting examples of polymers within the scope of the present invention can include polyacrylamide, polymethacrylate, polyethylene glycol, polyvinylpyrrolidone, hydroxypropylmethyl cellulose, hydroxypropyl cellulose, hydroxyethyl cellulose, methyl cellulose, carboxymethyl cellulose, cross-linked polyvinyl pyrrolidone, polyvinylpyrrolidone-vinyl acetate copolymer, polyvinylalcohol, polyacrylate, polyacrylic acid, cross-linked polyacrylate, cross-linked polyacrylic acid, polyethylene oxide, polyethylene glycol, polyvinylalkyl ether-maleic acid copolymer, carboxy vinyl polymer, polystyrene, and combination thereof. In some configurations, a polyvinylpyrrolidone can be a polyvinylpyrrolidone provided by International Specialty Products, Wayne, N.J. In some configurations, a hydroxypropylcellulose can be Klucel® hydroxypropylcellulose supplied by Hercules, Inc. Wilmington, Del. In some configurations, the synthetic polymer can comprise at least about 90%, at least about 95%, at least about 96%, at least about 97%, at least about 98%, at least about 99% or greater percent of one monomeric repeating unit.

In some configurations, the term “polymer” can also encompass polymerizable monomers and salts thereof. In these configurations, a polymer can comprise at least about 90%, at least about 95%, at least about 96%, at least about 97%, at least about 98%, at least about 99% or greater percent of a monomer.

As noted above, in certain embodiments, the synthetic polymer can be a polyacrylate. Non-limiting examples of polyacrylates of the present invention include polyacrylate, cross-linked polyacrylates, alkyl acrylates such as polymethacrylate, alicyclic, aromatic, heterocyclic and vinyl group-containing polyacrylates, hydroxy-containing polyacrylates, such as alkoxy polyacrylates, carboxylic acid-containing polyacrylates, polycyanoacrylates, polydialkylaminoethylacrylates, polyfluoroalkylacrylates polyethylacrylate, polybutylacrylate such as poly n-butylacrylate and poly t-butyl acrylate, poly 2-ethylhexylacrylate, polycyclohexylacrylate, polybenzylacrylate, poly 2-hydroxyethylacrylate, poly2-hydroxypropylacrylate, polydimethylaminoethylacrylate, polydiethylaminoethylacrylate, polyglycidylacrylate, polytetrahydrofurfuryl acrylate; poly alkylmethacrylates, such as polymethylmethacrylate, polyethylmethacrylate, poly di and tri methacrylates, alicyclic, aromatic, heterocyclic and vinyl group-containing polymethacrylates, hydroxy containing polymethacrylates such as poly alkoxymethacrylates, poly dialkylaminoethyl methacrylates, polyfluoroalkylmethacrylates, polybutylmethacrylate such as poly n-butylmethacrylate, poly t-butyl methacrylate, poly 2-ethylhexyl methacrylate, poly yclohexylmethacrylate, polybenzylmethacrylate, poly 2-hydroxyethyl methacrylate, poly 2-hydroxypropylmethacrylate, polydimethylaminoethylmethacrylate, polydiethylaminoethylmethacrylate, polyglycidylmethacrylate, and polytetrahydrofurfurylmethacrylate.

In some embodiments, the synthetic polymer can be a polyacrylamide or a substituted polyacrylamide. Non-limiting examples of polyacrylamides include polyacrylamide, cross-linked polyacrylamides, alkyl acrylamides such as polymethacrylamide, alicyclic, aromatic, heterocyclic and vinyl group-containing polyacrylamides, hydroxy-containing polyacrylamides, such as alkoxy polyacrylamides, carboxylic acid-containing polyacrylamides, polydialkylaminoethylacrylamides, polyfluoroalkylacrylamides, polyethylacrylamide, polybutylacrylamide, such as poly n-butylacrylamide and poly t-butylacrylamide, poly 2-ethylhexylacrylamide, polycyclohexylacrylamide, polybenzylacrylamide, poly 2-hydroxyethylacrylamide, poly 2-hydroxypropylacrylamide, polydimethylaminoethylacrylamide, polydiethylaminoethylacrylamide, polyglycidylacrylamide, polytetrahydrofurfurylacrylamide, alkyl methacrylamides, such as polymethylmethacrylamide, polyethylmethacrylamide, alicyclic, aromatic, heterocyclic and vinyl group-containing polymethacrylamides, hydroxy containing polymethacrylamides such as alkoxymethacrylamides, poly di and tri methacrylamides, polydialkylaminoethylmethacrylamides, polyfluoroalkyl methacrylamides, polybutylmethacrylamide such as poly n-butylmethacrylamide and t-butyl methacrylamide, poly 2-ethylhexyl methacrylamide, poly cyclohexylmethacrylamide, polybenzylmethacrylamide, poly 2-hydroxyethyl methacrylamide, poly 2-hydroxypropyl methacrylamide, polydimethylaminoethylmethacrylamide, polydiethylaminoethylmethacrylamide, polyglycidylmethacrylamide, and polytetrahydrofurfurylmethacrylamide.

As used herein, “polymer” can mean not only a polymer, but also a monomer that polymerizes in conjunction with application to a tooth. For example, in some embodiments, a tooth-coating composition can comprise cyanoacrylate monomer. Polymerization of the cyanoacrylate monomers can initiate upon contact with an aqueous fluid such as, for example, saliva on the surface of a tooth. Similarly, a tooth-coating composition can comprise an epoxy monomer which can polymerize upon contact of the composition with an initiator shortly before or after application to a tooth.

As noted above, in certain embodiments, the film-forming resin can be a natural resin. Non-limiting examples of natural resins within the scope of the present invention include shellacs, colophoniums, mastics or cellulose polymers. In certain configurations, the natural resin can be at least partially purified following extraction from its natural source. A purification can remove, for example, one or more colored contaminants, such as chromophoric impurities. Such purification can result in a tooth coating that is whiter than a comparable tooth coating in which impurities have not been removed from the natural resin. For example, a shellac that can comprise a tooth coating composition can be, for example, a Bulls Eye® shellac supplied by Zinsser Co., Inc. (Somerset, N.J.).

In various embodiments, the film-forming resin (either natural or synthetic polymer) used in a composition herein can have a weight average molecular weight, number average molecular weight, Z-average molecular weight or viscosity average molecular weight of from about 100 to about 5,000,000; from about 200 to about 2,500,000; from about 500 to about 1,000,000; from about 1,000 to about 500,000; or from about 10,000 to about 125,000.

In various embodiments, a tooth coating is a tooth-coating fluid. “Fluid,” as used herein, means a non-gaseous, non-solid substance. In various embodiments, the fluid can be a liquid or a semi-solid. The viscosity of a fluid can range from freely flowable, low viscosity to extremely high viscosity in which flow is slow or even imperceptible to an unaided human under ambient conditions. For example, ordinary silica glass, such as glass comprised by a window pane, can be considered a fluid herein. A fluid herein can be, in certain embodiments, a thixotropic liquid. A “semi-solid” as used herein can be a gel, a colloid, a paste, an ointment, or a gum. As used herein, semi-solids and liquids are fluids distinguished on the basis of viscosity: a semi-solid is a high viscosity fluid, while a liquid has lower viscosity. There is no definitive dividing line between these types of fluids. A fluid of a tooth-coating composition herein can have a viscosity, measured at 25° C., ranging from about 0.18 milliPascal-sec (mPa-s) to about 100,000,000 mPa-s, from about 0.5 mPa-s to about 10,000,000 mPa-s, from about 1 mPa-s to about 1,000,000 mPa-s, from about 1.5 mPa-s to about 100,000 mPa-s, or from about 2 mPa-s to about 10,000 Pa-s. In some configurations, a fluid of a composition herein can have a viscosity of from about 100 to about 1,000 Pa-s. Without being limited by theory, it is believed that the viscosity of a fluid herein will vary with the amount (percentage) of a film-forming resin component and the weight average molecular weight of a film-forming resin component.

In various embodiments, a tooth coating as described herein can be an adhesive fluid that adheres to teeth. Without being limited by theory, it is believed that the adhesiveness of the fluid of the present invention will vary with the amount (percentage) of the film-forming resin component and the weight average molecular weight of the film-forming resin component. Adhesiveness can be measured using standard adhesion tests known in the art, for example, the adhesive test disclosed in U.S. Pat. No. 6,613,812 to Bui. In certain embodiments, the adhesiveness between a tooth and a film formed from a fluid of the present invention can be from about at least 500 pounds per square inch (PSI), at least 1,000 PSI, at least 2,000 PSI, or greater.

In various embodiments of the present invention, a tooth coating comprising a film-forming resin can form a film on a tooth surface following its application thereon. In some embodiments, a formulation of a composition can comprise a film-forming resin and a volatile solvent which can dissolve the resin. The solvent can be, for example, an organic solvent, such as an alcohol, for example, ethanol. A composition can comprise a solvent in an amount of from about 1% (w/w) to about 70% (w/w), from about 10% (w/w) to about 50% (w/w), from about 20% (w/w) to about 40% (w/w), or from about 25% (w/w) to about 35% (w/w). In some embodiments, a film can form as the solvent is removed, for example through evaporation. In some embodiments, a film can form as monomers comprised by the composition polymerize. Without being limited by theory, it is believed that the longevity of a film, i.e., the period of time during which the film remains intact on the teeth, will vary with the percentage amount of the film-forming resin component in the film and the weight average molecular weight of the film-forming resin component.

In various embodiments, a skilled artisan can select a percentage amount and weight average molecular weight of a polymer comprised by a composition herein depending on the intended usage. For example, a user desiring to color his or her teeth for one evening can use a formulation comprising a low percentage of resin and/or a low average molecular weight resin, while a user desiring a tooth-coating that lasts several months can use a formulation comprising a high percentage of resin and/or a high average molecular weight resin.

In some embodiments, a tooth coating can comprise at least one colorant. The term “colorant” is used herein to describe a substance that can impart a color when applied to a tooth. A color, as used herein, can be any perceivable hue, tint, or shade, including but not limited to spectrum colors, colors comprised within the L*a*b* color space, colors comprised within the RGB color space, as well as black, brown, gray and white. A color imparted to a tooth by a tooth-coating fluid described herein can be a white color or a non-white color. In various embodiments, the colorant can be a pigment or dye. In certain embodiments, a pigment can be a white pigment that can impart a white color to a tooth.

In some configurations, the colorant can comprise a plurality of pigment particles. A pigment, as used herein, can mean a particulate colorant. In some configurations, the compositions of the present invention can comprise one or more colorants at a concentration, individually or in total, of from about 0.1% (w/w) up to about 20% (w/w), from about 1% (w/w) up to about 19% (w/w), from about 2% (w/w) up to about 18% (w/w), from about 3% (w/w) up to about 17% (w/w), from about 4% (w/w) up to about 16% (w/w), or from about 6% (w/w) up to about 15% (w/w). In some configurations, the pigment particles can have an average size of from about 0.1 micron to about 1000 microns, from about 0.3 micron to about 100 microns, or from about 0.5 microns to about 50 microns in diameter or longest dimension.

In various embodiments, the colorant can be a white pigment. The white pigment can be, for example, a white polymer. In some configurations, the white polymer can be a polymer such as described in U.S. Pat. No. 6,669,930 to Hoic. Non-limiting examples of such polmers include polyethylene, polypropylene, ethylene/propylene copolymer, polytetrafluoroethylene and polyhexafluoropropene. Specific examples of white polymers include polyethylene PE220, polypropylene, and polytetrafluoroethylene (PTFE), as supplied by PreSperse, Inc. (Somerset N.J.). Such white polymers can be high molecular weight polymers having weight average molecular weight, number average molecular weight, Z-average molecular weight, or viscosity average molecular weight of from about 100 to about 10,000,000; from about 200 to about 5,000,000; from about 500 to about 1,000,000; from about 1,000 to about 500,000; from about 10,000 to about 100,000, or from about 20,000 to about 50,000.

In some configurations, the white pigment can comprise a metal oxide such as, for example, titanium oxide, aluminum oxide, tin oxide, calcium oxide, magnesium oxide, zinc oxide or a combination thereof.

In various embodiments, a colorant can be a non-white colorant. In some configurations, the non-white colorant can comprise an organic pigment. In certain embodiments, the non-white colorant can be a colorant approved for incorporation into a food, drug or cosmetic by a regulatory agency, such as, for example, FD&C or D&C pigments and dyes approved by the FDA for use in the United States. Non-limiting examples of non-white colorants include FD&C Red No. 3 (sodium salt of tetraiodofluorescein), Food Red 17, 6-hydroxy-5-{(2-methoxy-5-methyl-4-sulphophenyl)azo}-2-naphthalenesulfonic acid, Food Yellow 13, mono sulphonic acid of quinophtalone, disulphonic acid of quinophtalone, monosulphonic acid of 2-(2-quinolyl) indanedione, disulphonic acid of 2-(2-quinolyl) indanedione, FD&C Yellow No. 5, FD&C Yellow No. 6, FD&C Green No. 3, FD&C Blue No. 1, FD&C Blue No. 2, D&C Red #30, phthalocyanine green, salts thereof and mixtures thereof.

In some embodiments, the non-white colorant can be a dye lake pigment. In some configurations, the dye lake can be a calcium or aluminum salt of an FD&C dye such as, for example, FD&C Green #1 lake, FD&C Blue #2 lake, D&C Red #30 lake or FD&C Yellow #15 lake, or mixtures thereof.

In various embodiments, the non-white colorant can comprise an inorganic pigment. Non-limiting examples of inorganic pigments can include certain metal oxide pigments such as for example, copper oxide, iron oxide and chromium oxide. Other non-white inorganic pigments that can be comprised by a tooth coating alone or in combination can be, for example, mineral pigments, such as ultramarine blue (lapis lazuli).

In certain configurations, the colorant can comprise a dye contained within a water-insoluble polymer. In non-limiting example, the dye FD&C Blue #1 can be contained within a water-insoluble polymer such as a polyethylene such as that found in polyethylene beads (e.g., Microblue Spectrabeads, Micropowders, Inc.) In certain embodiments, the colorant can be a non-bleeding dye.

In some embodiments, the tooth coating can comprise pearlescent particles. In some configurations, the pearlescent particles can provide a white pearlescent color. In other configurations, the pearlescent particles can provide a non-white pearlescent color. “Pearlescence” and “pearlescent,” as used herein, can refer to an optical property of a material in which the material has a pearl-like, lustrous appearance. A pearlescent material can provide a sense of depth and shine, and can be aesthetically appealing. Without being limited by theory, pearlescent particles are believed to partially reflect and partially refract incident light. The extent of partial refraction or reflection of incident light by a pearlescent material can depend on the angle of light incidence and/or the angle of viewing. As used herein, “pearlescence” can include iridescence and opalescence. Pearlescent materials can appear monochromatic or appear to change color depending on viewing angle.

Pearlescent particles used in the compositions herein can provide aesthetic or cosmetic effects such as, for example, sparkle or luster. In some configurations, a composition can comprise monochromatic pearlescent particles. In some configurations, a composition can comprise pearlescent particles presenting a variety of colors. A tooth-coating composition can comprise pearlescent particles from about 0.1% (w/w) up to about 20% (w/w), from about 1% (w/w) up to about 19% (w/w), from about 2% (w/w) up to about 18% (w/w), from about 3% (w/w) up to about 17% (w/w), from about 4% (w/w) up to about 16% (w/w), or from about 6% (w/w) up to about 15% (w/w). In some configurations, the pearlescent particles can have an average size of from about 0.5 micron to about 500 microns, from about 1 micron to about 100 microns, or from about 2 microns to about 20 microns in diameter or longest dimension.

Pearlescent particles can comprise a single mineral or chemical species, such as, for example a silicate such as mica, or bismuth oxychloride. By “mica” is meant any one of a group of hydrous aluminum silicate minerals with platy morphology and perfect basal (micaceous) cleavage. Mica can be, for example, sheet mica, scrap mica or flake mica, as exemplified by muscovite, biotite or phlogopite type micas. In some embodiments, the pearlescent particles can comprise a complex comprising more than one mineral or chemical species, such as, for example, mica coated with a metal oxide such as titanium oxide. In some embodiments, pearlescent particles can comprise at least one non-white component, such as iron oxide, or can comprise an optically interfering composition, for example as provided by certain iridescent or opalescent minerals such as opal, or compositions comprising mica coated with one or more layers of titanium oxide. Pearlescent particles can also be of biological origin, for example fish scale or mother-of-pearl. Certain pearlescent particles of biological origin can comprise calcium carbonate, such as, for example, pearl, mollusk shell such as mother-of-pearl obtained from oyster shell, or nacre.

In some embodiments, pearlescent particles can be for example those described as Timiron® pigments, Colorona® pigments, Dichrona® pigments, Soloron® pigments, Biron® powders, Biron® dispersions, Nailsyn® dispersions, or Microna® matte colors (all registered trademarks of EM Industries, Inc. Hawthorne, N.Y., division of E. Merck). For example, mica titanium particles can be pearlescent particles such as Timiron® particles. White or pearlescent mica titanium particles can be, for example “Silverwhite” Timiron® particles such as Timiron® Starluster MP-1 15, Timiron® Supersheen MP-1001, Timiron® Sparkle MP-47, Timiron® Supersilk MP-1005, Timiron® Pearl Flake MP-10, Timiron® Pearl Sheen MP-30, Timiron® Super Silver Fine, Timiron® Gleamer Flake MP- 111, Timiron® Ultraluster MP-45, Timiron® Transwhite MP-18, Timiron® Diamond Cluster MP-149, Timiron® Super Silver, Timiron® Stardust MP-80, Timiron® Arctic Silver or Timiron® Snowflake MP-99.

In some configurations, the mica particles can comprise mica and a metal oxide such as titanium oxide in a non-white in color, presenting, for example, an iridescent or interference based color. For example, a tooth coating can comprise a gold pigment, such as Timiron® Gold Plus MP-25, Timiron® Sun Gold Sparkle MP-29, Timiron® Fine Gold MP-20, Timiron® Transgold MP-28 or Timiron® Karat Gold MP-24; an interference pigment, such as Timiron® Super Green, Timiron® Super Blue, Timiron® Super Red, Timiron® Gold MP-127, Timiron® Blue MP-155, Timiron® Super Gold, Timiron® Gold Glow MP-26, Timiron® Silk Gold, Timiron® Silk Red, Timiron® Silk Blue, Timiron® Silk Green, Timiron® Super Copper, Timiron® Super Violet, Timiron® Starlight Red, Timiron® Starlight Blue, Timiron® Starlight Gold, Timiron® Starlight Green, Timiron® Splendid Gold, Timiron® Splendid Red, Timiron® Splendid Blue, Timiron® Splendid Green, Timiron® Splendid Copper or Timiron® Splendid Violet, or a metallic color pigment such as Timiron® MP-60 Bronze or Timiron® MP-65 Copper. In some configurations, a fluid can comprise multiple types of pigments for cosmetic or aesthetic effects,

In various embodiments, the present invention provides methods for coloring a tooth in a mammal. The mammal can be a human. The methods can comprise applying to a tooth, a tooth-coloring amount of a fluid comprising one or more cosmetic coloring agents and a film-forming resin. In some configurations, the film-forming resin can be a synthetic polymer as described herein or a natural resin as described herein. In some configurations, the synthetic polymer or natural resin can be at a concentration of at least about 10% (w/w), at least about 15% (w/w), at least about 20% (w/w), at least about 25% (w/w) or 26% (w/w) or at least about 30% (w/w). In some configurations, the natural resin can be at a concentration of at least 25% (w/w) or 26% (w/w) or at least about 30% (w/w). The tooth coating comprising one or more cosmetic coloring agents and a synthetic polymer can be a tooth-coating fluid as described herein. In some configurations, the coloring agents can comprise one or more white and/or pearlescent coloring agents. In some configurations, the white or pearlescent coloring agent can comprise titanium oxide, mica titanium, or a white polymer as described herein. In some configurations, the application of a fluid of the present invention to a tooth can result in a perceivable increase in tooth whiteness. The whiteness of a tooth that has been coated with a tooth-coating composition can be determined visually by comparison with the Vita Shade Guide scale of whiteness, or measured by a skilled artisan using a color measurement instrument such as a Minolta CR-321 chromometer. For example, a tooth to which the fluid has been applied can exhibit an increase in its Vita Guide shade of at least one increment, for example, from A1 to B1. In addition, the presence of white pigment in a composition can result in a tooth that is brighter than B1 on the Vita Shade Guide scale of whiteness.

Application of a fluid composition of the present invention can be accomplished using methods known in the art. For example, an applicator such as a brush can be dipped in a tooth-coating fluid described herein, and the fluid can then be painted onto a tooth. In addition to brush application, other non-limiting modes of application can comprise applying a rinse comprising a tooth-coating fluid, applying a semi-solid tooth-coating fluid from a stick resembling a lipstick, applying a semi-solid form using a crayon-like stick, spraying on the fluid, dabbing on the fluid using a towelette, or transferring the fluid from adhesive strip. Following application of a fluid of the invention to targeted teeth, a tooth-adherent film can form comprising a resin, a colorant and pearlescent particles. Adhesion of a film to a tooth can be promoted by allowing the fluid to dry following application to the tooth. In some embodiments, a film forms once the fluid dries or a polymer forms. A film once formed can remain on the tooth for at least about one hour to about one year, from about one day to about six months, from about one week to about three months, or from about two weeks to about two months. A film formed on a tooth can be removed, for example, through friction, e.g., as provided by tooth brushing or mechanical scraping, or, in some embodiments, through application of a solvent, such as, for example, ethanol. In addition, in some configurations, application of a composition of the invention to a tooth can have a therapeutically beneficial effect, even without the presence of a therapeutic active in the composition, as the composition will prevent acids present in the oral cavity from contacting the tooth.

In certain embodiments, application of a fluid of the present invention to teeth requires no special equipment or training; for example, the fluid can be self-applied by an individual user, or applied by an esthetician. In some configurations, prior to application to targeted teeth, the teeth can be cleaned, e.g., through brushing, to promote good adhesion of the fluid. Alternatively, a dental professional such as a dental hygienist or a dentist can clean the targeted teeth more thoroughly using professional equipment and methods prior to fluid application. Immediately following application, solvent comprised by the applied fluid can be removed, e.g., through evaporation, or polymerization of monomers to form polymer can be allowed to occur. In some configurations, a tooth-coating composition that is expected to endure for an extended period, for example, for six months after application, can be applied by a dental professional such as a dentist or a dental hygienist.

In various embodiments, a composition of the invention can further comprise one or more active ingredients. An active ingredient can be, for example, a therapeutic active, a bleaching agent, an anti-caries agent, a tartar control agent, an anti-plaque agent, a periodontal active, an anti-sensitivity agent, an anti-microbial agent, or a breath freshening agent. Some non-limiting examples of active materials are described in U.S. Pat. No. 6,596,298 to Leung et al., and in U.S. patent application Ser. No. 10/739,803 to Boyd, et al., filed Dec. 18, 2003.

In some configurations, a bleaching agent can be, in non-limiting example, a peroxide, a hypochlorite, a peroxy acid, a metal chlorite, a perborate, a percarbonate such as sodium percarbonate, a persulfate such as an oxone as disclosed in U.S. Pat. No. 6,419,906 to Xu et al., or an oxidoreductase. A peroxide compound can be, for example, hydrogen peroxide, urea peroxide, or calcium peroxide. A metal chlorite can be, for example, calcium chlorite, barium chlorite, magnesium chlorite, lithium chlorite, sodium chlorite, or potassium chlorite. An oxidoreductase can be, for example, an enzyme disclosed in U.S. Pat. No. 5,989,526 to Aaslyng et al., such as a laccase, an oxidase or a peroxidase.

In various configurations, the tooth-coating fluid of the present invention can further comprise one or more additional components, such as, in non-limiting examples, a hydrophobic organic non-polymeric material, an alcohol, a sweetener, and/or a flavorant. The hydrophobic organic non-polymeric material can be, for example, a wax such as a beeswax, for example a white beeswax, or a paraffin. In various embodiments, the composition can comprise a hydrophobic non-polymeric material at a level of from about 0.01% to about 2% (w/w). A sweetener can be a natural or synthetic sweetener. In certain configurations, the sweetener can be a carbohydrate, such as, for example, xylose, ribose, glucose (dextrose), mannose, galactose, fructose (levulose), sucrose or maltose. In certain configurations, the sweetener can be a non-carbohydrate sweetener such as a saccharin salt, for example a sodium or a calcium salt of saccharin; a cyclamate salt; or a dipeptide based sweetener, such an L-aspartic acid derived sweetener such as L-aspartyl-L-phenylalanine methyl ester (aspartame). In various embodiments, the composition can comprise a sweetener at a level of from about 0.01% to about 2% (w/w). In certain configurations, a flavorant can be, for example, a synthetic flavor oil or a flavoring aromatic, an oleo resin or an extract derived from plants, leaves, flowers, fruits, or a combination thereof. Non-limiting examples of flavor oils include spearmint oil, cinnamon oil, peppermint oil, clove oil, bay oil, thyme oil, cedar leaf oil, oil of nutmeg, oil of sage, and oil of bitter almonds. Flavorants can be used individually or in admixture. Commonly used flavors include mints such as peppermint, artificial vanilla, cinnamon derivatives, and various fruit flavors. In certain embodiments, a flavorant can be a flavorant described in Chemicals Used in Food Processing, publication 1274 by the National Academy of Sciences, pages 63-258. In various embodiments, the composition can comprise a flavorant at a level of from about 0.01% to about 2% (w/w).

In some configurations, an anti-caries agent can be, in non-limiting example, a fluoride, a stannous salt, polyvinylphosphonic acid (PVPA) or an anti-caries polymer such as a polycationic polymer described in U.S. Pat. No. 5,063,046 to Grollier.

In some configurations, a tartar control agent can be, in non-limiting example, a dialkali or tetraalkali metal pyrophosphate salt such as Na₄P₂O₇, K₄P₂O₇, Na₂K₂P₂O₇, Na₂H₂P₂O₇ and K₂H₂P₂O₇; a long chain polyphosphate such as sodium hexametaphosphate; and a cyclic phosphate such as sodium trimetaphosphate. In some configurations, a polyphosphate can be β-phase calcium pyrophosphate, such as disclosed in U.S. Pat. No. 6,241,974 to White, Jr.

In some configurations, an anti-sensitivity agent can be, in non-limiting example, a desensitizing salt such as potassium nitrate, a desensitizing polymer, or a combination thereof.

In some configurations, an anti-microbial agent can be, for example, triclosan, cetyl pyridium chloride, domiphen bromide, a quaternary ammonium salt, a zinc compound, sanguinarine, an anti-microbial fluoride, alexidine, octonidine, EDTA, thymol, methyl salicylate, eucalyptol, menthol or a combination thereof.

In some configurations, a breath-freshening agent can be an odor-reducing agent. An odor reducing agent can be a sulfur precipitating agent. Non-limiting examples of sulfur-precipitating agents include metal salts, such as a copper salt and a zinc salt. Non-limiting examples of such salts include copper gluconate, zinc citrate and zinc gluconate.

In certain embodiments, the present invention provides methods for enhancing whole body health. It is expected that whole body health can be promoted by administrating a composition of the present invention to at least one tooth within an oral cavity. In various such embodiments a composition of the invention comprises a functional material. In certain configurations, the functional material is an oral care active or a therapeutic active. In one embodiment, the functional material can provide a health benefit that is non-exclusive to oral health, for example a functional material can contribute to the amelioration, risk reduction, treatment or prevention of disease, dysfunction, or other abnormality. Such materials include those described in PCT Publication WO 02/02128 A2 to Doyle et al.

Certain embodiments of the present invention can be illustrated by the following examples.

### EXAMPLE 1

This Example illustrates a composition of the invention comprising a natural resin with white and pearlescent pigments. In this example, a fluid comprising a mixture of the following substances can be provided in the designated amounts, as shown in Table 1:

TABLE 1

| Substance | Amount (percentage w/w) |
|---|---|
| ethanol 96% | 20.35 |
| white beeswax | 0.35 |
| colophonium | 23.35 |
| sodium saccharin | 0.50 |
| mastic | 8.61 |
| shellac | 15.67 |
| flavor | 0.47 |
| TiO₂ | 14 |
| Timiron ® | 16.7 |

### EXAMPLE 2

This example illustrates a method of making the composition described in Example 1.

In this example, titanium dioxide in an amount of 14% (w/w) and a Timiron® mica titanium in an amount of 16.7% (w/w) are admixed with “Duraphat® Placebo” (Colgate-Palmolive Co.) at 69.3 % (w/w). Duraphat® Placebo is the tooth-adherent oral pharmaceutical sodium fluoride product Duraphat® prepared without sodium fluoride. The composition of Duraphat® Placebo is provided in Table 2.

TABLE 2

| Substance | Amount (percentage w/w) |
|---|---|
| ethanol 96% | 29.36 |
| white beeswax | 0.51 |
| colophonium | 33.7 |
| sodium saccharin | 0.72 |
| mastic | 12.42 |
| shellac | 22.61 |
| flavor | 0.68 |

### EXAMPLE 3

This example illustrates an formulation for a resin mixture alternative to Duraphat® Placebo.

As shown in Table 3, Duraphat® Placebo, of the formulation presented in Table 2, can be replaced with a mixture of the formulation presented in Table 3. This formulation is expected to provide increased whiteness to the final composition (and to a user's teeth) compared to Duraphat® Placebo because a natural resin (shellac), which can be amber in color, is replaced with a colorless synthetic polymer (polyacrylate).

TABLE 3

| Substance | Amount (percentage w/w) |
|---|---|
| ethanol 96% | 29.36 |
| white beeswax | 0.51 |
| colophonium | 33.7 |
| sodium saccharin | 0.72 |
| mastic | 12.42 |
| polyacrylate | 22.61 |
| flavor | 0.68 |

### INDUSTRIAL APPLICATION

The tooth coating and tooth whitening fluids of the present invention which provide therapeutically active ingredients can be used advantageously for prophylaxis and therapy of, for example, oral conditions or diseases, as well as promotion of whole-body health. The latter can include a reduction in risk of development of systemic diseases, such as cardiovascular disease, stroke, diabetes, severe respiratory infection, premature and low birth weight infants (including associated post-partum dysfunction in neurologic/developmental function), and associated increased risk of mortality. Thus, in various embodiments, the invention can provide for use of a natural or synthetic resin, colorant particles and pearlescent particles in the manufacture of a tooth-coating fluid that imparts a whitening or a color to the teeth. The invention can also provide for the use of a therapeutic active for the manufacture of a medicament for the treatment or prevention of an oral condition. Such oral conditions include, for example, the presence of caries, oral sensitivity, tartar, gingivitis, oral malodour, or a microbial infection. In addition, in various embodiments, the invention can provide for use of a tooth whitening product comprising a therapeutic active for the manufacture of a medicament for promotion of whole-body health.

As various changes could be made in the above methods and compositions without departing from the scope of the invention, it is intended that all matter contained in the above description be interpreted as illustrative and not in a limiting sense. Unless explicitly stated to recite activities that have been done (i.e., using the past tense), illustrations and examples are not intended to be a representation that given embodiments of this invention have, or have not, been performed.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinency of the cited references.

1. A tooth coating comprising at least one film-forming synthetic polymer at a concentration of at least 10% (w/w), a colorant, and pearlescent particles.

2. A tooth coating according to claim 1, wherein the tooth coating is selected from group consisting of a tooth-coating fluid, a veneer, a natural hard dental surface, an artificial hard dental surface, a crown, a restorative veneer, a partial denture, and a composite.

3. A tooth coating according to claim 1, wherein the synthetic polymer is selected from the group consisting of polyacrylates, polyacrylamides, polyvinylpyrollidones, polyvinylalcohols, polyethylene glycols, polyethylene oxides, polystyrenes, hydroxypropylmethyl celluloses, hydroxypropyl celluloses, hydroxyethyl celluloses, methyl celluloses, carboxymethyl celluloses, copolymers, block copolymers, graft copolymers, cross-linked polymers and combinations thereof.

4. A tooth coating according to claim 1, wherein the colorant comprises one or more types of white particles which are independently comprised of a material selected from the group consisting of titanium oxide, aluminum oxide, tin oxide, calcium oxide, magnesium oxide, zinc oxide, polyethylene, polypropylene, ethylene/propylene copolymer, polytetrafluoroethylene, and polyhexafluoropropene.

5. A tooth coating according to claim 1, wherein the colorant comprises one or more types of non-white particles which are independently comprised of a material selected from the group consisting of copper oxide, chromium oxide, ferric oxide, ultramarine blue, phthalocyanine green, FD&C Green No. 1 lake, FD&C Blue No. 2 lake, D&C Red No. 30 lake, FD&C Yellow No. 15 lake, FD&C Red No. 3, D&C Red No. 30, Food Red No. 17, disodium salt of 6-hydroxy-5-{(2-methoxy-5-methyl-4-sulphophenyl)azo}-2-naphthalenesulfonic acid, Food Yellow No. 13, sodium salt of a mixture of the mono and disulphonic acids of quinophtalone or 2-(2-quinolyl) indanedione, FD&C Yellow No. No. 5, FD&C Yellow No. 6, FD&C Green No. 3, FD&C Blue No. 1, FD&C Blue No. 2, and combinations thereof.

6. A tooth coating according to claim 1, wherein the pearlescent particles comprise one or more types of particles which are independently comprised of a material selected from the group consisting of bismuth oxychloride, nacre, mollusk shell, fish scale, pearl, silicates selected from the group consisting of mica, opal and silica, and combinations thereof.

7. A tooth coating according to claim 6, wherein the pearlescent particles further comprise a metal oxide coating wherein the metal oxide is selected from the group consisting of titanium oxide, iron oxide, aluminum oxide, tin oxide, copper oxide, calcium oxide, magnesium oxide, barium oxide, chromium oxide and combinations thereof.

8. A tooth coating according to claim 7, wherein the pearlescent particles further comprise a protective coating.

9. A tooth coating according to claim 7, wherein the pearlescent particles are mica titanium particles.

10. A tooth coating according to claim 9, wherein the colorant is titanium dioxide and the pearlescent particles are mica titanium.

11. A tooth coating according to claim 10, wherein the colorant is titanium dioxide, the pearlescent particles are mica titanium and the synthetic polymer is a polyacrylate.

12. A tooth coating according to claim 11, wherein the titanium oxide particles are at a concentration of at least about 1% (w/w) and the pearlescent particles are at a concentration of at least about 3% (w/w).

13. A tooth coating according to claim 1, further comprising an active ingredient selected from the group consisting of anti-caries agents, anti-sensitivity agents, anti-microbial agents, bleaching agents, and combinations thereof.

14. A tooth coating comprising at least one film-forming resin, a colorant at a concentration of at least about 1% (w/w), and pearlescent particles at a concentration of at least about 3% (w/w).

15. A tooth coating according to claim 14, wherein the tooth coating is selected from group consisting of a tooth-coating fluid, a veneer, a natural hard dental surface, an artificial hard dental surface, a crown, a restorative veneer, a partial denture, and a composite.

16. A tooth coating according to claim 14, wherein the film-forming resin is a synthetic resin, a natural resin or a combination thereof, wherein the synthetic resin is selected from the group consisting of polyacrylates, polyacrylamides, polyvinylpyrollidones, polyvinylalcohols, polyethylene glycols, polyethylene oxides, polystyrenes, hydroxypropyl methyl celluloses, hydroxypropyl celluloses, hydroxyethyl celluloses, methyl celluloses, carboxymethyl celluloses, copolymers, block copolymers, graft copolymers cross-linked polymers and combinations thereof, and the natural resin is selected from the group consisting of colophoniums, mastics, shellacs, natural celluloses, and combinations thereof.

17. A tooth coating according to claim 14, wherein the colorant comprises one or more types of white particles which are independently comprised of a material selected from the group consisting of titanium oxide, aluminum oxide, tin oxide, calcium oxide, magnesium oxide, zinc oxide, polyethylene, polypropylene, ethylene/propylene copolymer, polytetrafluoroethylene, and polyhexafluoropropene.

18. A tooth coating according to claim 14, wherein the colorant comprises one or more types of non-white particles which are independently comprised of a material selected from the group consisting of copper oxide, chromium oxide, ferric oxide, ultramarine blue, phthalocyanine green, FD&C Green No. 1 lake, FD&C Blue No. 2 lake, D&C Red No. 30 lake, FD&C Yellow No. 15 lake, FD&C Red No. 3, D&C Red No. 30, Food Red No. 17, disodium salt of 6-hydroxy-5-{(2-methoxy-5-methyl-4-sulphophenyl)azo}-2-naphthalenesulfonic acid, Food Yellow No. 13, sodium salt of a mixture of the mono and disulphonic acids of quinophtalone or 2-(2-quinolyl) indanedione, FD&C Yellow No. No. 5, FD&C Yellow No. 6, FD&C Green No. 3, FD&C Blue No. 1, FD&C Blue No. 2, and combinations thereof.

19. A tooth coating according to claim 14, wherein the pearlescent particles comprise one or more types of particles which are independently comprised of a material selected from the group consisting of bismuth oxychloride, nacre, mollusk shell, fish scale, pearl, silicates selected from the group consisting of mica, opal and silica, and combinations thereof.

20. A tooth coating according to claim 19, wherein the pearlescent particles further comprise a metal oxide coating wherein the metal oxide is selected from the group consisting of titanium oxide, iron oxide, aluminum oxide, tin oxide, copper oxide, calcium oxide, magnesium oxide, barium oxide, chromium oxide and combinations thereof.

21. A tooth coating according to claim 20, wherein the pearlescent particles further comprise a protective coating.

22. A tooth coating according to claim 20, wherein the pearlescent particles are mica titanium particles.

23. A tooth coating according to claim 22, wherein the colorant is titanium dioxide and the pearlescent particles are mica titanium.

24. A tooth coating according to claim 21, wherein the colorant is titanium dioxide, the pearlescent particles are mica titanium and the synthetic polymer is a polyacrylate.

25. A tooth coating according to claim 24, wherein the titanium oxide particles are at a concentration of at least about 1% (w/w) and the pearlescent particles are at a concentration of at least about 3% (w/w).

26. A tooth coating according to claim 14, further comprising an active ingredient selected from the group consisting of anti-caries agents, anti-sensitivity agents, anti-microbial agents, bleaching agents, and combinations thereof.

27. A method of coloring a tooth in a mammal, the method comprising applying to the tooth a tooth-coloring amount of a tooth coating comprising at least one film-forming synthetic polymer at a concentration of at least 10% (w/w), a colorant, and pearlescent particles.

28. A method according to claim 27, wherein the tooth coating is selected from group consisting of a tooth-coating fluid, a veneer, a natural hard dental surface, an artificial hard dental surface, a crown, a restorative veneer, a partial denture, and a composite.

29. A method according to claim 27, wherein the colorant comprises one or more types of white particles which are independently comprised of a material selected from the group consisting of titanium oxide, aluminum oxide, tin oxide, calcium oxide, magnesium oxide, zinc oxide, polyethylene, polypropylene, ethylene/propylene copolymer, polytetrafluoroethylene, and polyhexafluoropropene.

30. A method according to claim 27, wherein the colorant comprises one or more types of non-white particles which are independently comprised of a material selected from the group consisting of copper oxide, chromium oxide, ferric oxide, ultramarine blue, phthalocyanine green, FD&C Green No. 1 lake, FD&C Blue No. 2 lake, D&C Red No. 30 lake, FD&C Yellow No. 15 lake, FD&C Red No. 3, D&C Red No. 30, Food Red No. 17, disodium salt of 6-hydroxy-5-{(2-methoxy-5-methyl-4-sulphophenyl)azo}-2-naphthalenesulfonic acid, Food Yellow No. 13, sodium salt of a mixture of the mono and disulphonic acids of quinophtalone or 2-(2-quinolyl) indanedione, FD&C Yellow No. No. 5, FD&C Yellow No. 6, FD&C Green No. 3, FD&C Blue No. 1, FD&C Blue No. 2, and combinations thereof.

31. A method according to claim 27, wherein the pearlescent particles comprise one or more types of particles which are independently comprised of a material selected from the group consisting of bismuth oxychloride, nacre, mollusk shell, fish scale, pearl, silicates selected from the group consisting of mica, opal and silica, and combinations thereof.

32. A method according to claim 27, wherein the pearlescent particles are further comprise a metal oxide coating wherein the metal oxide is selected from the group consisting of titanium oxide, iron oxide, aluminum oxide, tin oxide, copper oxide, calcium oxide, magnesium oxide, barium oxide, chromium oxide and combinations thereof.

33. A method according to claim 32, wherein the pearlescent particles are mica titanium particles.

34. A method according to claim 33, wherein the colorant is titanium dioxide and the pearlescent particles are mica titanium.

35. A method according to claim 34, wherein the colorant is titanium dioxide at a concentration of at least about 1% (w/w), the pearlescent particles are mica titanium at a concentration of at least about 3% (w/w) and the synthetic polymer is a polyacrylate.

36. A method according to claim 27, wherein the tooth coating further comprises an active ingredient selected from the group consisting of anti-caries agents, anti-sensitivity agents, anti-microbial agents, bleaching agents, and combinations thereof.

37. A method of coloring a tooth in a mammal, the method comprising applying to the tooth a tooth-coloring amount of a tooth coating comprising at least one film-forming resin, a colorant at a concentration of at least about 1% (w/w), and pearlescent particles at a concentration of at least about 3% (w/w).

38. A method according to claim 37, wherein the tooth coating is selected from group consisting of a tooth-coating fluid, a veneer, a natural hard dental surface, an artificial hard dental surface, a crown, a restorative veneer, a partial denture, and a composite.

39. A method according to claim 37, wherein the film-forming resin is a synthetic resin, a natural resin or a combination thereof, wherein the synthetic resin is selected from the group consisting of polyacrylates, polyacrylamides, polyvinylpyrollidones, polyvinylalcohols, polyethylene glycols, polyethylene oxides, polystyrenes, hydroxypropyl methyl celluloses, hydroxypropyl celluloses, hydroxyethyl celluloses, methyl celluloses, carboxymethyl celluloses, copolymers, block copolymers, graft copolymers cross-linked polymers and combinations thereof, and the natural resin is selected from the group consisting of colophoniums, mastics, shellacs, natural celluloses, and combinations thereof.

40. A method according to claim 37, wherein the colorant comprises one or more types of white particles which are independently comprised of a material selected from the group consisting of titanium oxide, aluminum oxide, tin oxide, calcium oxide, magnesium oxide, zinc oxide, polyethylene, polypropylene, ethylene/propylene copolymer, polytetrafluoroethylene, and polyhexafluoropropene.

41. A method according to claim 37, wherein the colorant comprises one or more types of non-white particles which are independently comprised of a material selected from the group consisting of copper oxide, chromium oxide, ferric oxide, ultramarine blue, phthalocyanine green, FD&C Green No. 1 lake, FD&C Blue No. 2 lake, D&C Red No. 30 lake, FD&C Yellow No. 15 lake, FD&C Red No. 3, D&C Red No. 30, Food Red No. 17, disodium salt of 6-hydroxy-5-{(2-methoxy-5 -methyl-4-sulphophenyl)azo}-2-naphthalenesulfonic acid, Food Yellow No. 13, sodium salt of a mixture of the mono and disulphonic acids of quinophtalone or 2-(2-quinolyl) indanedione, FD&C Yellow No. No. 5, FD&C Yellow No. 6, FD&C Green No. 3, FD&C Blue No. 1, FD&C Blue No. 2, and combinations thereof.

42. A method according to claim 37, wherein the pearlescent particles comprise one or more types of particles which are independently comprised of a material selected from the group consisting of bismuth oxychloride, nacre, mollusk shell, fish scale, pearl, silicates selected from the group consisting of mica, opal and silica, and combinations thereof.

43. A method according to claim 37, wherein the pearlescent particles further comprise a metal oxide coating wherein the metal oxide is selected from the group consisting of titanium oxide, iron oxide, aluminum oxide, tin oxide, copper oxide, calcium oxide, magnesium oxide, barium oxide, chromium oxide and combinations thereof.

44. A method according to claim 43, wherein the pearlescent particles are mica titanium particles.

45. A method according to claim 44, wherein the colorant is titanium dioxide and the pearlescent particles are mica titanium.

46. A method according to claim 45, wherein the colorant is titanium dioxide at a concentration of at least about 1% (w/w), the pearlescent particles are mica titanium at a concentration of at least about 3% (w/w) and the synthetic polymer is a polyacrylate.
